(19) United States
(12) Patent Application Publication
(10) Pub. No.: US 2015/0282755 A1
(43) Pub. Date: Oct. 8, 2015

(54) SYSTEM AND METHOD FOR DETECTING SEIZURE ACTIVITY

(71) Applicant: KING FAHD UNIVERSITY OF PETROLEUM AND MINERALS, DHAHRAN (SA)

(72) Inventors: MOHAMED DERICHE, DHAHRAN (SA); MOHAMMED ABDUL AZEEM SIDDIQUI, HYDERABAD (IN)

(73) Assignee: KING FAHD UNIVERSITY OF PETROLEUM AND MINERALS, DHAHRAN (SA)

(21) Appl. No.: 14/243,626

(22) Filed: Apr. 2, 2014

(51) Int. Cl. A61B 5/00; A61B 5/0476; A61B 5/0402

(52) U.S. Cl. CPC A61B 5/4094; A61B 5/0402; A61B 5/0476

(57) ABSTRACT

The system and method for detecting seizure activity combines signal traces from both an electroencephalogram (EEG) and an electrocardiogram (ECG) in order to detect and predict a seizure event in a patient. Determination of a seizure classification of the combination is based on Dempster-Shafer Theory (DST) to calculate a combined probability belief. Prior to combination, classification of the EEG and ECG data is performed by linear discriminant analysis (LDA) or naïve Bayesian classification to provide a seizure event classification or a non-seizure event classification.

### BACKGROUND OF THE INVENTION

1. Field of the Invention

The present invention relates to seizure detection and prediction, and particularly to a system and method for detecting seizure activity using a combination of electroencephalogram (EEG) and electrocardiogram (ECG) data from a patient.

2. Description of the Related Art

Seizures pose a great health risk due to both direct and indirect damage to the sufferer. Seizure disorders are the most common class of nervous system disorders, and there is evidence to suggest that being prone to seizures decreases life expectancy. Seizures may affect people throughout their entire lifetimes. Almost 6% of low birth weight infants and approximately 2% of all newborns admitted in neonatal intensive care units (ICUs) suffer from seizures. Additionally, it is estimated that about 2% of adults have had a seizure at some time in their lives.

Although seizures on their own rarely result in a fatality, seizures greatly impact the quality of a sufferer's life, and can also easily contribute to accidental death and injury. Up to 75% of adults suffering from seizures have reported suffering from depression and have been found to be at greater risk for suicide. In addition to outwardly obvious seizures, sufferers may also experience so-called “silent” seizures, which do not have any outward physical symptoms, but which can result in brain damage. Thus, there is an obvious need for detection of seizures at an early stage in order to prevent damage to the body or brain.

One problem in seizure detection is in the misinterpretation of other unrelated conditions as being seizure-related. Various neurological disorders may result in a patient exhibiting jerky movements, twitches or the like, which may be easily misinterpreted as a seizure. Unfortunately, in such situations, patients are often administered multiple antiepileptic drugs (AEDs) over periods of several days. Such patients tend to remain sedated in a hospital for relatively long periods of time due this false diagnosis.

Although electroencephalograms (EEGs) are used as a tool for the early detection of seizures, an accurate seizure diagnosis requires a specialist to correctly interpret the EEG data. Detection of seizures can be difficult, even for professionals. Even a trained neurologist may be fooled during visual inspection due to myogenic artifacts. FIG. 2A illustrates a sample EEG signal for a non-seizing patient. FIG. 2B shows a sample EEG signal for a patient with seizure traces. Although various algorithms for automatic detection of seizures based on EEG data have been developed, EEG-based systems and methods may miss a large percentage of seizures, specifically because seizures may also be associated with changes in heart beat rhythm and respiration rate; i.e., effects that are not based solely in the brain. Complex seizures can result from variations in cardiac rhythms, which would not be predicted in an EEG-based system.

Although there has been some work on using electrocardiograms (ECGs) for seizure detection, a complete and accurate detection method would need to combine the data from both an EEG and an ECG, allowing prediction for both brain-based and cardiovascular-based seizures. Previous approaches related to the combination of ECG and EEG data were based on various fusion techniques for decision-making based on the Bayesian formulation. However, such approaches did not provide meaningful solutions, since the Bayesian formulation of decision-making assumes a Boolean phenomenon, which leads to over-commitment; i.e., the degree of belief we have in the existence of a certain hypothesis. Thus, a small degree of belief in a certain hypothesis automatically leads to a large degree of belief in the negation of the hypothesis. To avoid such problems, it is necessary to develop a new technique for fusing information from EEG and ECG data without over-commitment. It would be desirable to be able to use the theory of evidence to fuse information from two independent classifiers, namely, one based on EEG signal analysis and the second based on the analysis of an ECG signal, to provide an accurate overall predictor for seizures.

Thus, a system and method for detecting seizure activity solving the aforementioned problems is desired.

### SUMMARY OF THE INVENTION

The system and method for detecting seizure activity combines signal traces from both an electroencephalogram (EEG) and an electrocardiogram (ECG) in order to detect and predict a seizure event in a patient. Determination of a seizure classification from the combination is based on Dempster-Shafer Theory (DST) to calculate a combined probability belief. Prior to combination, classification of the EEG and ECG data is performed by linear discriminant analysis (LDA) or naïve Bayesian classification to provide a seizure event classification or a non-seizure event classification.

The method for detecting seizure activity begins with the training of a neural network or the like with ECG and EEG feature vectors representing seizure event classification or non-seizure event classification. The EEG signal is represented in a time-frequency domain and a time-frequency representation matrix is generated therefrom. Singular value decomposition is applied to the time-frequency representation matrix to compute left and right singular vectors and a singular value matrix. A set of probability mass functions is then extracted from the singular value matrix, and a histogram is generated having 17 bins for the left singular vector for a first singular value.

The ECG signal is filtered and corrected for baseline wander to produce a filtered and baseline wander corrected ECG signal. R, P, Q, S and T wave peaks in the filtered and baseline wander corrected electrocardiogram signal are then determined, such that the following features may be extracted and calculated: an R-R interval mean (a mean value between consecutive R wave peaks in the filtered and baseline wander corrected electrocardiogram signal), an R-R interval variance (a variance between consecutive R wave intervals), a P height mean (a mean value of P wave peaks), a P-R duration (a duration between consecutive P and R wave peaks), and a Q-T duration (a duration between consecutive Q and T wave peaks in the filtered and baseline wander-corrected electrocardiogram signal).

An electroencephalogram classifier is applied to the histogram to calculate an electroencephalogram probability of a seizure classification, and an electrocardiogram classifier is applied to a feature dataset including the R wave peak, the P, Q, S, and T wave peaks, the R-R interval mean, the R-R interval variance, the P height mean, the P-R duration and the Q-T duration to calculate an electrocardiogram probability of a seizure classification. Classification of the EEG and ECG data is performed by linear discriminant analysis (LDA) or naïve Bayesian classification to provide a seizure event classification or a non-seizure event classification.

The electroencephalogram probability of a seizure classification and the electrocardiogram probability of a seizure classification are then combined using Dempster-Shafer Theory (DST) to determine a Dempster-Shafer belief. If the Dempster-Shafer belief has a probability value above a threshold value of ½, then the presence of a seizure event is indicated.

These and other features of the present invention will become readily apparent upon further review of the following specification.

### BRIEF DESCRIPTION OF THE DRAWINGS

FIG. 1 is a schematic diagram illustrating the major components in a system for detecting seizure activity according to the present invention.

FIG. 2A is an exemplary electroencephalogram (EEG) signal tracing for a non-seizing patient.

FIG. 2B is an exemplary electroencephalogram (EEG) signal tracing for a patient, showing seizure traces.

FIG. 3 is a graph showing energies of singular values of a time-frequency representation (TM) of a sample EEG signal.

FIG. 4A is a histogram generated from the probability mass function of a left singular vector corresponding to a first singular value of a first data sample of an EEG trace when a seizure is present generated by a method for detecting seizure activity according to the present invention.

FIG. 4B is a histogram generated from the probability mass function of a left singular vector corresponding to a first singular value of a first data sample of an EEG trace when a seizure is absent generated by the method for detecting seizure activity according to the present invention.

FIG. 4C is a histogram generated from the probability mass function of a right singular vector corresponding to the first singular value of the first data sample of an EEG trace when a seizure is present, generated by the method for detecting seizure activity according to the present invention (FIG. 4C is the right singular vector corresponding to the trace of FIG. 4A).

FIG. 4D is a histogram generated from the probability mass function of the right singular vector corresponding to the first singular value of the first data sample of an EEG trace when no seizure is present, generated by the method for detecting seizure activity according to the present invention (FIG. 4D is the right singular vector corresponding to the trace of FIG. 4B).

FIG. 5A is a histogram generated from the probability mass function of a left singular vector corresponding to a first singular value of a second data sample of an EEG trace when a seizure is present generated by a method for detecting seizure activity according to the present invention.

FIG. 5B is a histogram generated from the probability mass function of a left singular vector corresponding to a first singular value of a second data sample of an EEG trace when a seizure is absent generated by the method for detecting seizure activity according to the present invention.

FIG. 5C is a histogram generated from the probability mass function of a right singular vector corresponding to the first singular value of the second data sample of an EEG trace when a seizure is present, generated by the method for detecting seizure activity according to the present invention (FIG. 5C is the right singular vector corresponding to the trace of FIG. 5A).

FIG. 5D is a histogram generated from the probability mass function of the right singular vector corresponding to the first singular value of the second data sample of an EEG trace when no seizure is present, generated by the method for detecting seizure activity according to the present invention (FIG. 5D is the right singular vector corresponding to the trace of FIG. 5B).

FIG. 6A is a histogram generated from the probability mass function of a left singular vector corresponding to a second singular value of the first data sample of an EEG trace when a seizure is present, generated by the method for detecting seizure activity according to the present invention.

FIG. 6B is a histogram generated from the probability mass function of a left singular vector corresponding to a second singular value of a first data sample of an EEG trace when a seizure is absent generated by the method for detecting seizure activity according to the present invention.

FIG. 6C is a histogram generated from the probability mass function of a right singular vector corresponding to the second singular value of the first data sample of an EEG trace when a seizure is present, generated by the method for detecting seizure activity according to the present invention (FIG. 6C is the right singular vector corresponding to the trace of FIG. 6A).

FIG. 6D is a histogram generated from the probability mass function of the right singular vector corresponding to the second singular value of the first data sample of an EEG trace when no seizure is present, generated by the method for detecting seizure activity according to the present invention (FIG. 6D is the right singular vector corresponding to the trace of FIG. 6B).

FIG. 7A is a histogram generated from the probability mass function of a left singular vector corresponding to a second singular value of a second data sample of an EEG trace when a seizure is present generated by a method for detecting seizure activity according to the present invention.

FIG. 7B is a histogram generated from the probability mass function of a left singular vector corresponding to a second singular value of a second data sample of an EEG trace when a seizure is absent generated by the method for detecting seizure activity according to the present invention.

FIG. 7C is a histogram generated from the probability mass function of a right singular vector corresponding to the second singular value of the second data sample of an EEG trace when a seizure is present, generated by the method for detecting seizure activity according to the present invention (FIG. 7C is the right singular vector corresponding to the trace of FIG. 7A).

FIG. 7D is a histogram generated from the probability mass function of the right singular vector corresponding to the second singular value of the second data sample of an EEG trace when no seizure is present, generated by the method for detecting seizure activity according to the present invention (FIG. 7D is the right singular vector corresponding to the trace of FIG. 7B).

FIG. 8A is a histogram generated from the probability mass function of a left singular vector of a data sample of an EEG trace when a seizure is present, generated by the method for detecting seizure activity according to the present invention.

FIG. 8B is a histogram generated from the probability mass function of a left singular vector of a data sample of the EEG trace of FIG. 8A but time delayed for 10 seconds, generated by the method for detecting seizure activity according to the present invention.

FIG. 8C is a histogram generated from the probability mass function of a right singular vector of the data sample of FIG. 8A.

FIG. 8D is a histogram generated from the probability mass function of the right singular vector of the data sample of FIG. 8A, but time delayed for 10 seconds.

FIGS. 9A, 9B, 9C, and 9D illustrate a wavelet-transformed electrocardiogram (ECG) signal at increasing scales of 2¹, 2², 2³ and 2⁴, respectively.

FIG. 10 is a sample ECG signal for use in the method for detecting seizure activity according to the present invention.

FIG. 11 is the ECG signal of FIG. 10 following filtering and correction for baseline wander.

FIG. 12A is a Level 4 wavelet transformed ECG signal of the ECG signal of FIG. 11.

FIG. 12B illustrates identification of the P, Q, R, S and T wave peaks in the filtered and baseline wander corrected ECG signal of FIG. 11 based upon identification of the R wave from the Level 4 wavelet transform of FIG. 12A.

FIG. 13 is a graph showing the accuracy of seizure detection using the present method for detecting seizure activity for an EEG dataset using a linear discriminant analysis (LDA) classifier.

FIG. 14 is a graph showing the accuracy of seizure detection using the present method for detecting seizure activity for the EEG dataset of FIG. 13, using a naïve Bayesian classifier.

FIG. 15 is a graph showing the accuracy of seizure detection using the present method for detecting seizure activity for an ECG dataset using a linear discriminant analysis (LDA) classifier.

FIG. 16 is a graph showing the accuracy of seizure detection using the present method for detecting seizure activity for the ECG dataset of FIG. 15, using a naïve Bayesian classifier.

FIG. 17 is a block diagram illustrating system components of a controller for implementing the method for detecting seizure activity according to the present invention.

Unless otherwise indicated, similar reference characters denote corresponding features consistently throughout the attached drawings.

### DETAILED DESCRIPTION OF THE PREFERRED EMBODIMENTS

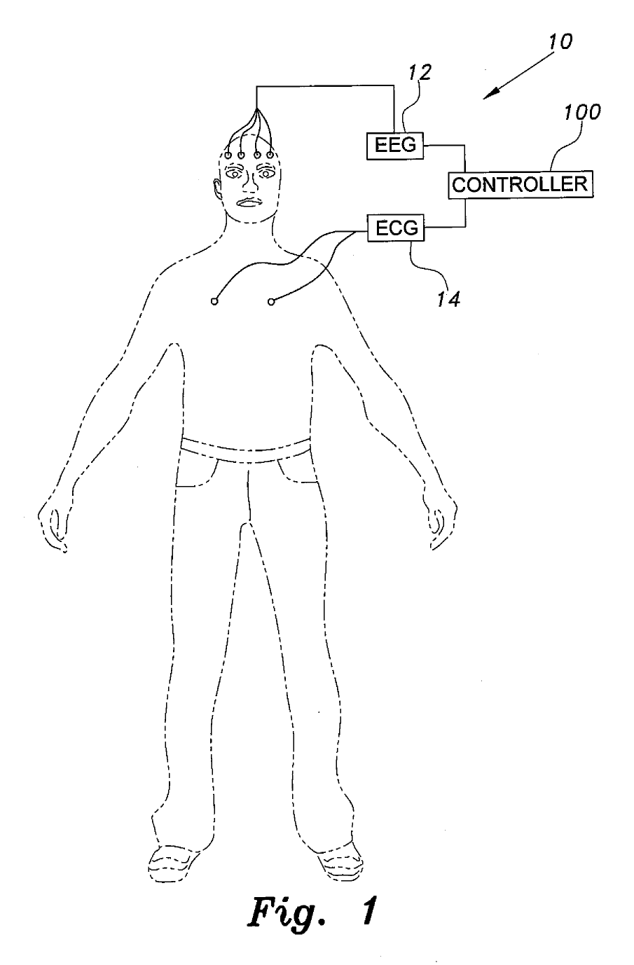

The system and method for detecting seizure activity combines signal traces from both an electroencephalogram (EEG) and an electrocardiogram (ECG) in order to detect and predict a seizure event in a patient. Determination of a seizure classification of the combination is based on Dempster-Shafer Theory (DST) to calculate a combined probability belief. Prior to combination, classification of the EEG and ECG data is performed by linear discriminant analysis (LDA) or naïve Bayesian classification to provide a seizure event classification or a non-seizure event classification. As diagrammatically illustrated in FIG. 1, signals are obtained from the patient by both an EEG 12 and an ECG 14. It should be understood that any suitable type of EEG or ECG may be used in system 10. These signals are fed to controller 100, which performs classification and combination, as will be described in detail below.

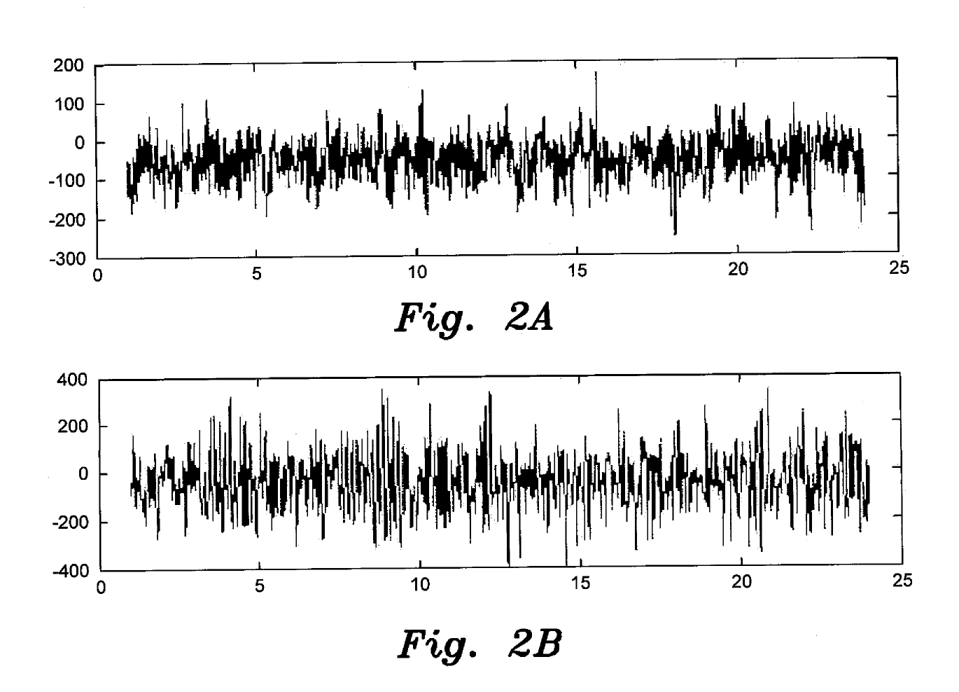

The electroencephalogram (EEG) signal, in its unmodified form, such as those illustrated in FIGS. 2A and 2B, does not show any information related to the frequency content of the signal. In order to get information from non-stationary signals, such as these EEG signals, the time-frequency representation must be used. Since the time-frequency representations cannot necessarily give high resolution in both the time and frequency domains at the same time, the selection of a particular time-frequency representation depends on the particular type of application and the specific features of interest. In order to find the optimal time-frequency representation for the EEG signal, the EEG signal representation was tested under different time-frequency distributions. Specifically, four different time-frequency distribution for the representation of EEG signal were tested, including the Short Time Fourier Transform (STFT), the Wigner-Ville Time-Frequency Representation (WV-TFR), the Choi-Williams Time-Frequency Representation (CW-TFR), and the Zhao-Atlas-Marks Time-Frequency Representation (ZAM-TFR). Each representation was tested for both a seizure trace and a corresponding non-seizure trace. From this comparison, it was determined that STFT and the Wigner-Ville distribution gave poor representations of the seizure trace. The Choi-Williams representation was also found to give a poor time resolution, particularly when compared to the Zhao-Atlas-Marks Time-Frequency Representation (ZAM-TFR). Further, the ZAM-TFR was found to show several lines in the range between 0 Hz and 4 Hz that were not found using the other TFRs. Thus, it was determined that the ZAM-TFR distribution should be used. As will be described in detail below, once the EEG trace is represented using ZAM-TFR, a Singular Value Decomposition (SVD) will be performed on the TFR matrix to extract the signal information from the time-frequency matrix.

The Zhao-Atlas-Marks Time-Frequency Representation (ZAM-TFR) is a cone-shaped distribution function and one of the members of Cohen's class distribution functions. In the ZAM-TFR, the kernel function φ(t,τ) for time t in the τ domain is given by φ=gₑ(τ)rect(t/τ) or φ(t,τ)=gₒ(τ)rect(t/τ), where the function gₑ(τ) is a general, even, bounded, real function. For unbounded gₑ(τ), the kernel becomes Cohen's Born-Jordan kernel. The function gₒ(τ) is a general, odd, bounded, imaginary function, e.g., gₒ(τ)=−jsgn(τ)gₑ(τ). For gₒ(τ)=−jsgn(τ), this kernel maximally concentrates interference terms to occur only at signal frequencies, and preserves finite-frequency support.

For the above, the original EEG signal is 23.6 seconds long with a sampling rate of 178.13 Hz. For training, 4,097 samples were used. The original EEG signal was then down-sampled to 28 Hz to reduce the computational load, corresponding to 1,024 samples. The down-sampled EEG signal is then transformed to the time-frequency matrix using 500 bins. Thus, the matrix size representing the time-frequency matrix is 500×1,024.

Singular Value Decomposition (SVD) is a common factorization approach of rectangular real or complex matrices. The basic objective of SVD is to find a set of “typical” patterns that describe the largest amount of variance in a given dataset. In the present method, SVD is used on the time-frequency distribution matrix X (M×N):

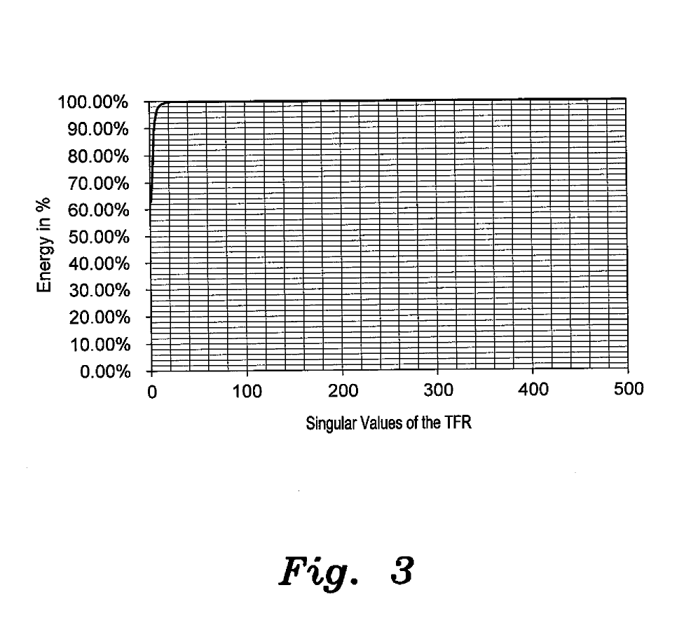

X=UΣV^{T} (1)

where U (M×M) and V (N×N) are orthonormal matrices, and E is an M×N diagonal matrix of singular values (σᵢⱼ≠0 if i=j and σ₁₁≧σ₂₂≧ . . . ≧0). The columns of orthonormal matrices U and V are called the left and right singular vectors (SV), respectively. It should be noted that matrices U and V are mutually orthogonal. The singular values (σᵢⱼ) represent the importance of individual SVs in the composition of the matrix. The SVs corresponding to larger singular values provide more information about the structure of patterns contained in the data. As shown in FIG. 3, the first singular value contains more than 60% of the energy of the signal. Thus, only the first singular vector corresponding to the first singular value is used as a feature vector for differentiating between the seizure and non-seizure traces. In the above, the U matrix is 500×500 (M×M), representing the frequency information, and the size of the V matrix is 1,204×1,204 (N×N), representing the time information.

Following singular value decomposition, feature vector extraction is performed. As noted above, the singular values are orthonormal. Thus, they have unit norms, and their squared elements can be treated as probability mass functions (PMFs) for different elements of the vector. For example, the PMF of the first columns of matrix U can be given as:

Fᵤ={u₁₁²,u₁₂², . . . ,u_{1N}²}. (2)

From the above obtained PMFs, the histogram bins can then be computed. The entire column data of the left singular vector is distributed in non-linear histogram bins. Non-linear histogram bins are used to focus more on the low frequency and high frequency information of the signal, since seizure events are related to activity in the delta region (0 Hz to 4 Hz). It should be noted that first vectors of the U matrix and the V matrix correspond to the first singular value of the Σ matrix. Since the columns of the U and V matrices are orthonormal, the square of the elements can be considered to be PMFs. Thus, by taking the square of individual elements of the first vectors of the matrices U and V corresponding to the first singular value of the Σ matrix, one obtains the vectors U₁(1:500) and V₁(1:1024), where U₁(1:500)={u₁₁², u₁₂², . . . , u_{1M}²} and V₁(1:1024)={v₁₁², v₁₂², . . . , v_{1M}²}.

The histogram used in the present method for the left singular vector has 17 bins, which represent the frequency content of the signal. Experiments with varying bins sizes were performed. A bin size of 17 bins was found to be the most useful with a non-linear distribution of frequency information for classification purposes. The values of the PMFs in the U₁(1:500) vector are summed at irregular intervals and are distributed in the 17 histogram bins such that they represent the 0-14 Hz range of the EEG signal in a non-linear way, placing emphasis on the lower 0-4 Hz and the 12-14 Hz ranges of the EEG signal. The first four histogram bins represent information of the respective frequency ranges 0.5-1.0 Hz, 1.0-2.0 Hz, 2.0-3.0 Hz, and 3.0-4.0 Hz. These histogram bins represent the characteristic vector to be fed to the linear discriminant network for discriminating a seizure event. In a similar manner, the column data for the right singular vector is also distributed in histogram bins. However, uniform bins are used in this case, since the right singular vector represents the information related to time. Thus, there is no need to distribute the data in a non-linear manner. In the present method, 10 bins are used to represent the time information.

With regard to time-frequency-based seizure feature extraction from an EEG signal, the EEG signal is first filtered such that any activity above 14 Hz is filtered by passing the signal through a low pass filter with a cut-off frequency of 14 Hz. The filtered signal is then down-sampled. In our experiments, the EEG readings were each 23.6 seconds long, having a sample rate of 178.13 Hz. A total of 4,097 samples were used. The sampling rate was reduced to 28 Hz in order to reduce the computational load. Following the Nyquist rate, this sampling rate is enough to analyze signals with frequencies less than 14 Hz.

Following down-sampling, the Zhao-Atlas-Marks (ZAM) distribution is used to represent the EEG signal in the time-frequency domain and generate a time-frequency representation matrix. Singular value decomposition is then applied to the time-frequency representation matrix to compute left and right singular vectors and a singular value matrix. Since the columns of the singular value matrix are orthonormal, the square of the elements of the matrix can be considered as probability mass functions (PMFs), as in equation (2) above.

Table 1 below shows how each of the 17 histogram bins represents the summation of part of the vector U₁(1:500). With regard to the right singular vector of the histogram, since the right singular vector represents the time signal, the PMFs in the V₁(1:1024) vector are summed at regular intervals and are distributed in 10 histogram bins such that they represent the 0-23.5 seconds time interval with regular intervals, as shown in Table 2 below.

TABLE 1
The 17 Histogram Bins of the Left Singular Vector

| Bin No. | Summation | Representing (Hz) |
|---|---|---|
| 1 | Sum(U₁(1: 28)) | 0-0.8 |
| 2 | Sum(U₁(29: 50)) | 0.8-1.4 |
| 3 | Sum(U₁(51: 71)) | 1.4-2.2 |
| 4 | Sum(U₁(72: 108)) | 2.2-3.2 |
| 5 | Sum(U₁(109: 138)) | 3.2-4.0 |
| 6 | Sum(U₁(139: 175)) | 4.0-5.0 |
| 7 | Sum(U₁(176: 212)) | 5.0-6.0 |
| 8 | Sum(U₁(213: 245)) | 6.0-7.0 |
| 9 | Sum(U₁(246: 282)) | 7.0-8.0 |
| 10 | Sum(U₁(283: 318)) | 8.0-9.0 |
| 11 | Sum(U₁(319: 354)) | 9.0-10.0 |
| 12 | Sum(U₁(355: 390)) | 10.0-11.0 |
| 13 | Sum(U₁(391: 426)) | 11.0-12.0 |
| 14 | Sum(U₁(427: 444)) | 12.0-12.5 |
| 15 | Sum(U₁(445: 462)) | 12.5-13.0 |
| 16 | Sum(U₁(461: 480)) | 13.0-13.5 |
| 17 | Sum(U₁(481: 500)) | 13.5-14.0 |

TABLE 2
The 10 Histogram Bins of the Right Singular Vector

| Bin No. | Summation | Representing (Seconds) |
|---|---|---|
| 1 | Sum(V₁(1: 102)) | 0-2.36 |
| 2 | Sum(V₁(103: 205)) | 2.36-4.72 |
| 3 | Sum(V₁(206: 308)) | 4.72-7.02 |
| 4 | Sum(V₁(309: 411)) | 7.02-9.44 |
| 5 | Sum(V₁(412: 514)) | 9.44-11.8 |
| 6 | Sum(V₁(515: 617)) | 11.8-14.16 |
| 7 | Sum(V₁(618: 720)) | 14.16-16.52 |
| 8 | Sum(V₁(721: 823)) | 16.52-18.88 |
| 9 | Sum(V₁(824: 924)) | 18.88-21.24 |
| 10 | Sum(V₁(924: 1024)) | 21.24-23.6 |

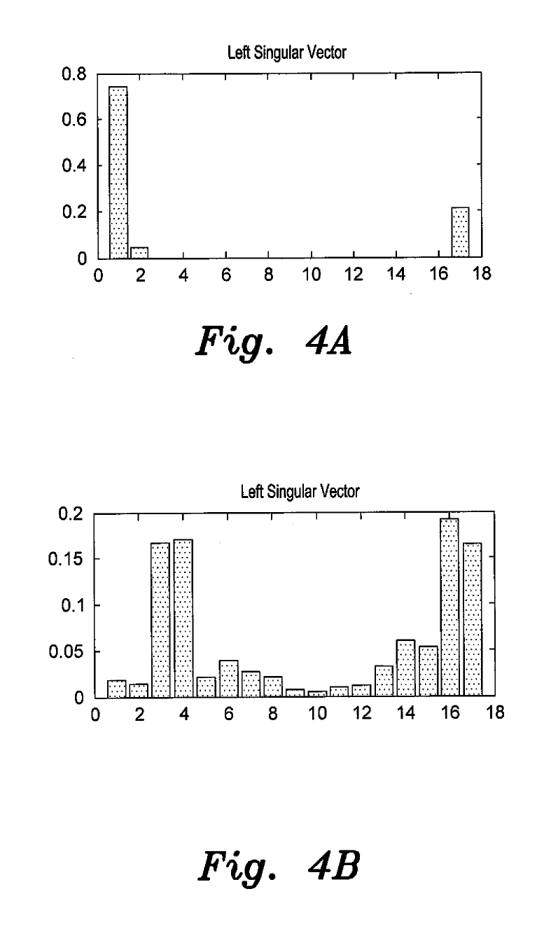

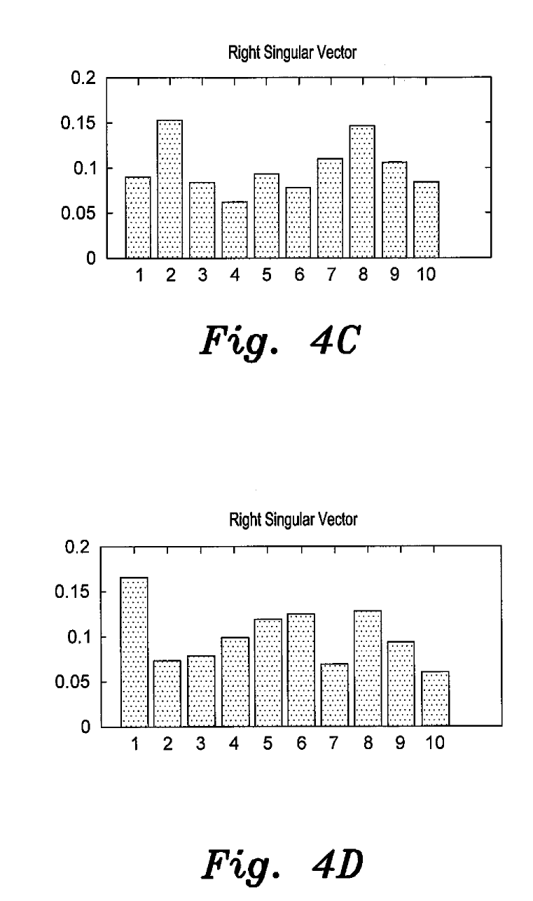

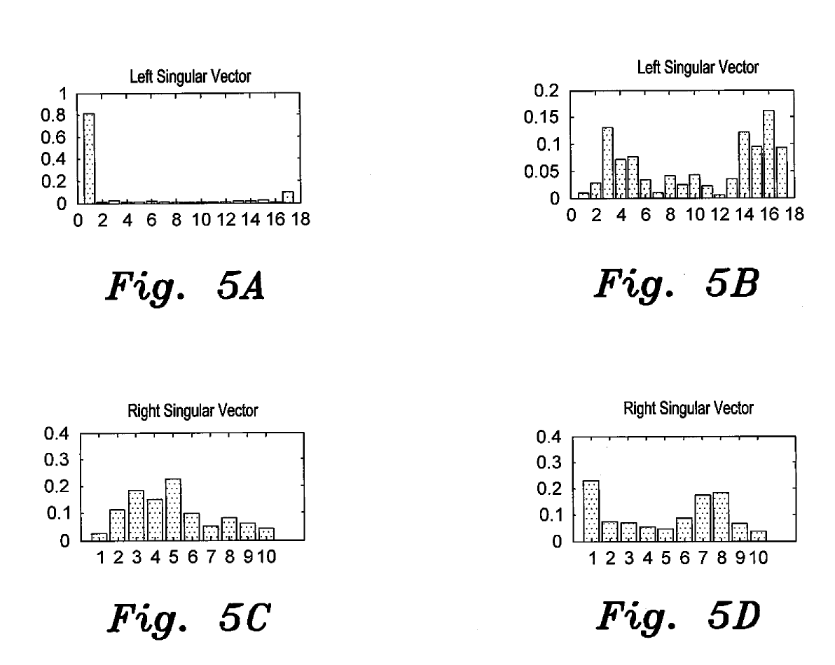

From the probability mass functions, histograms are generated with, respectively, 17 bins for the left singular vector and 10 bins for the right singular vector. FIGS. 4A and 4B are histograms generated for a first data sample for the left singular vector (i.e., a seizure trace) and FIGS. 4C and 4D are histograms generated for the same first data sample for the right singular vector, each corresponding to the first singular value. FIGS. 4A and 4C correspond to a trace where a seizure was present, and FIGS. 4B and 4D correspond to a trace where no seizure was present. Similarly, FIGS. 5A and 5B are histograms generated for a second data sample for the left singular vector, and FIGS. 5C and 5D are histograms generated for the same second data sample for the right singular vector, each corresponding to the first singular value. FIGS. 5A and 5C correspond to a trace where a seizure was present, and FIGS. 5B and 5D correspond to a trace where no seizure was present. It can be clearly seen that the histograms corresponding to the left singular vectors easily discriminate between seizure and non-seizure events (FIGS. 4A (seizure), 4B (non-seizure), 5A (seizure), and 5B (non-seizure)). For a seizure trace (FIGS. 4A, 5A), the first and last bins of the histogram have relatively large values and the remainder of the bins are almost zero, whereas for a non-seizure trace (FIGS. 4B, 5B), the histogram bins are unevenly distributed. Hence, the histogram bins of the left singular vector corresponding to the first singular value are used as the feature vector. The histogram bins for the right singular vector relate to the time values and are distributed in linear manner. Thus, they do not contribute to distinguishing between a seizure trace and a non-seizure trace.

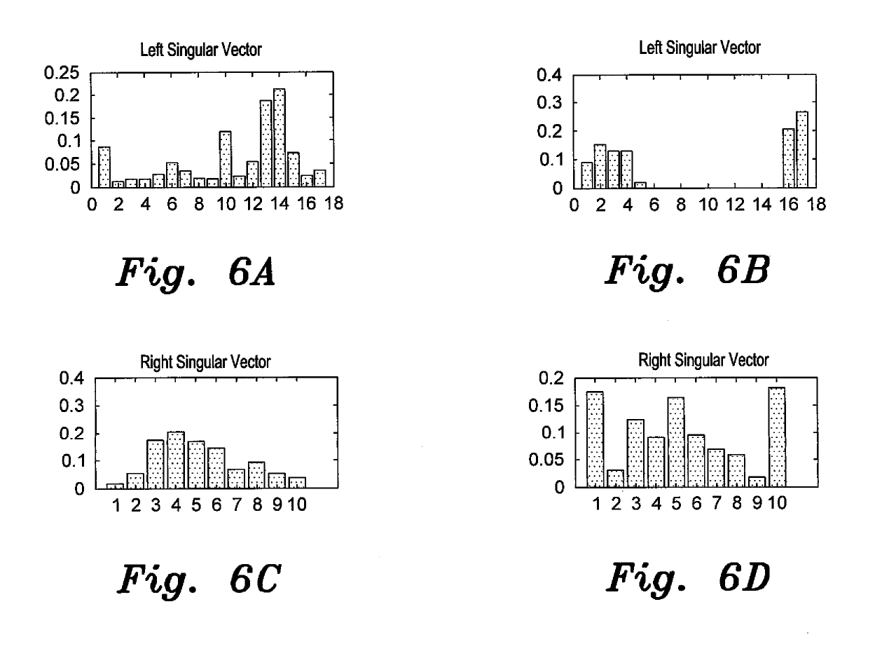

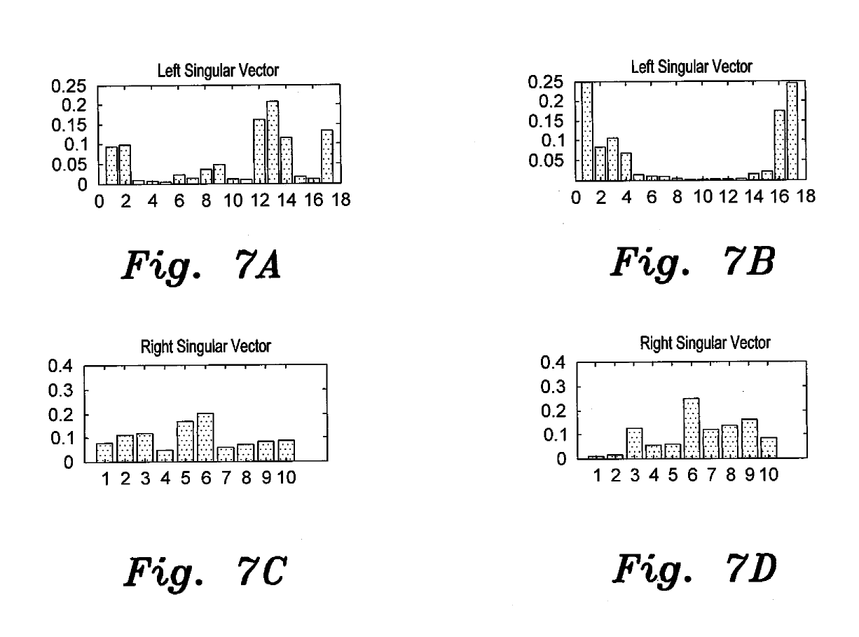

FIGS. 6A and 6B are histograms generated for the first data sample of FIGS. 4A-4D for the left singular vector, and FIGS. 6C and 6D are histograms generated for the same first data sample for the right singular vector, each corresponding to the second singular value. FIGS. 6A and 6C relate to traces where seizure was present, and FIGS. 6B and 6D relate to traces where seizure was absent. Similarly, FIGS. 7A and 7B are histograms generated for the second data sample of FIGS. 5A-5D for the left singular vector, and FIGS. 7C and 7D are histograms generated for the same second data sample for the right singular vector, each corresponding to the second singular value. FIGS. 7A and 7C relate to traces where seizure was present, and FIGS. 7B and 7D relate to traces where seizure was absent. As can be seen in FIGS. 6A and 7A, the left singular vector, representing a seizure trace, is unevenly distributed. Thus, the use of singular vectors from singular values other than the first singular value reduces overall accuracy. Thus, the present method uses only the histogram bins of the left singular vector corresponding to the first singular value as the feature vector.

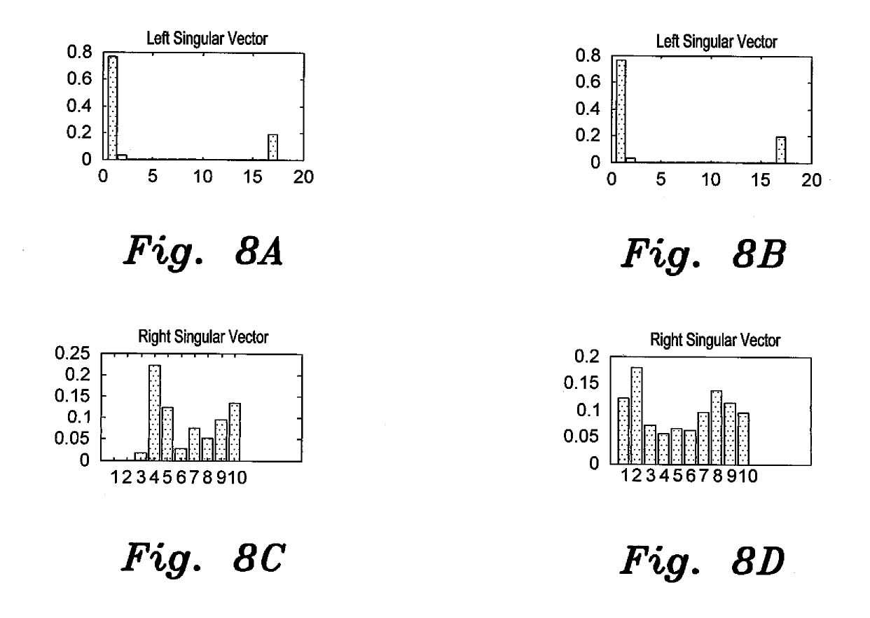

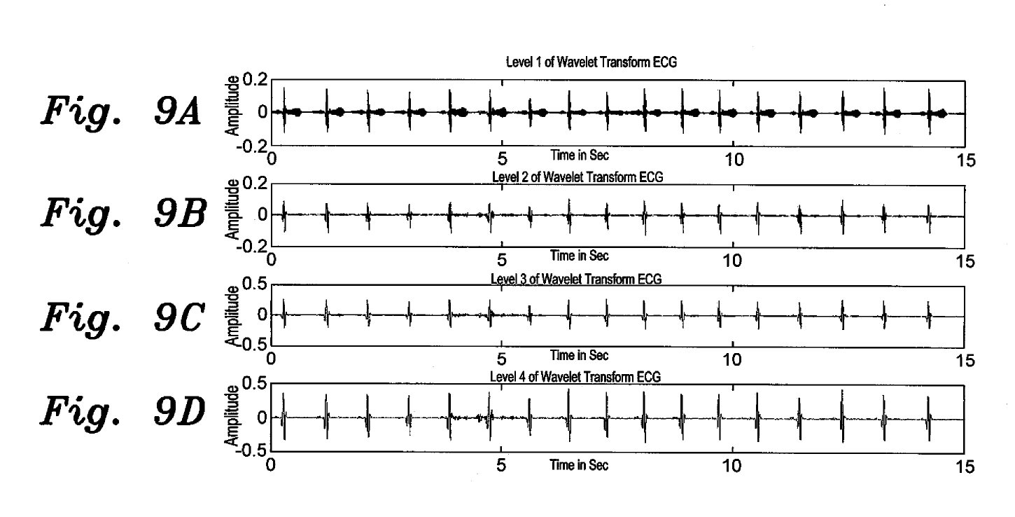

Further, the right singular vector only shows the time information of the signal, i.e., the right singular vector only shows the information at the instant of time when the seizure occurred. However, a seizure can occur at different instants of time for different patients, and even at different times for the same patient. To emphasize this point, FIGS. 8A and 8B show histograms for a left singular vector for a patient undergoing a seizure. FIGS. 8C and 8D show the histograms for the right singular vector. FIGS. 8B and 8D show the signal time-delayed (i.e., shifted) by ten seconds. Both signals undergo the same steps for extracting the features. It can be seen that the left singular value (FIGS. 8A and 8B) of both signals remains the same, but there is a change in the right singular value of the two signals (FIGS. 8C and 8D) due to the time shift. Thus, the use of the right singular value in discriminating the signals for detecting seizures is misleading and should be avoided. The final feature set for the present EEG-based part of the method uses the 17 bins of the histogram representing the left singular vector corresponding to the first singular value. This feature set is used for training the classification algorithm, as will be described in greater detail below, to identify the pattern of seizure and non-seizure events.

The “QRS complex” is a name for the combination of three of the graphical deflections seen on a typical electrocardiogram (ECG). It is usually the central and most visually obvious part of the tracing. The QRS complex corresponds to the depolarization of the right and left ventricles of the human heart. In adults, it normally lasts 0.06-0.10 seconds, and in children and during physical activity, it may be shorter. Typically, an ECG has five deflections, arbitrarily named “P” through “T” waves. The Q, R, and S waves occur in rapid succession, do not all appear in all leads, and reflect a single event, and thus are usually considered together. A Q wave is any downward deflection after the P-wave. An R wave follows as an upward deflection, and the S wave is any downward deflection after the R wave. The T-wave follows the S-wave, and in some cases an additional U wave follows the T wave. With regard to the ECG portion of data used in the present method, five separate features of the ECG are used; the R-R interval mean (where the R-R interval is the interval between one R wave and the next R wave); the R-R interval variance; the P height mean; the P-R duration; and the Q-T duration.

In order to extract the R-R interval from the ECG signal, as well as the other P, Q, S, and T waves, the ECG signal is decomposed using the conventional wavelet transform. The ECG signal is decomposed into four scales, ranging from 2¹ to 2⁴. It was found that the wavelet transform at small scales reflects the high frequency components of the signal, and at large scales, the low frequency components. The energy contained at certain scales depends on the center frequency of the wavelet used.

The 2⁴ scale of the wavelet-transformed ECG signal is used to detect the R-peak because most energies of a typical QRS complex are at scales 2³ and 2⁴. It was found that high frequency noise, such as that from electric line interference, muscle activity, electromagnetic interference and the like, is concentrated in the lower scales of 2¹ and 2², while the levels 2³ and 2⁴ contribute less noise compared to the lower scales. Thus, the frequency of the QRS complex is mainly present in the 2³ and 2⁴ scales. Since the 2⁴ scale is found to have less noise compared to 2³, the present method uses the 2⁴ scale for extracting R peaks. The wavelet-decomposed ECG signal is shown in FIGS. 9A-9D. The R peaks are then extracted from the 2⁴ scale by setting some threshold. Once the R peaks are extracted, the P, Q, S and T peaks are then extracted from the ECG wave using the well-known Tompkins method, as will be described in greater detail below.

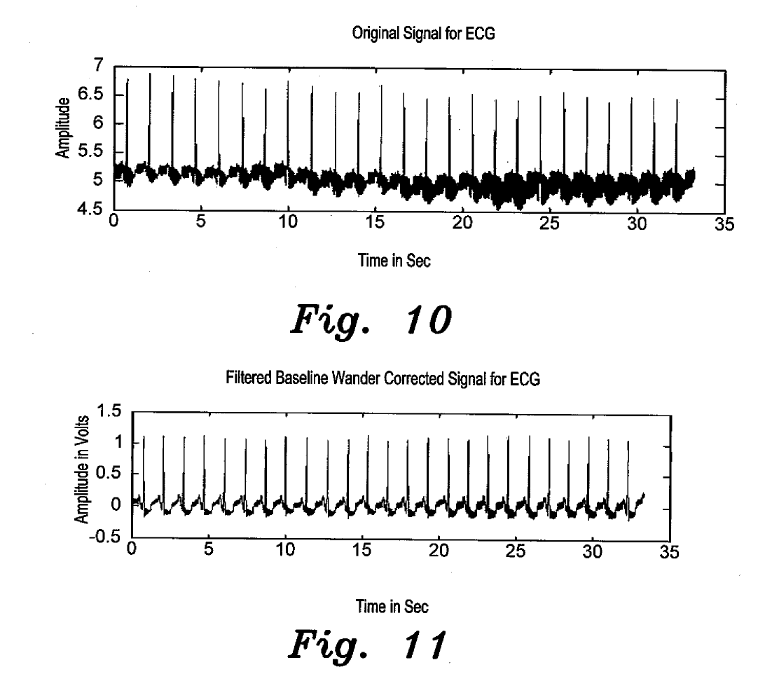

For ECG feature extraction, an ECG signal of 60 second duration is used. An original (i.e., non-filtered) ECG signal sample is shown in FIG. 10. The data consists of numerous artifacts and noise due to the presence of power line interference, bowel movements (also called “EGG movement”), muscle activity, and electromagnetic interference. Thus, in order to remove this noise, the ECG signal is pre-processed using a conventional finite impulse response (FIR) filter.

Baseline wandering is also considered as an artifact which affects the measuring of ECG parameters. The respiration and electrode impedance change due to perspiration and increased body movements are the main causes of baseline wandering. In order to remove baseline wandering, the filtered signal is passed through a median filter of 200 ms duration that removes the QRS complexes. The filtered signal is again passed through a median filter of 600 ms duration to remove the T wave. The filtered signal obtained in this step is then subtracted from the filtered signal obtained in the previous step (i.e., the FIR filtered signal), which gives the baseline wander eliminated signal. The filtered and baseline wander corrected signal is shown in FIG. 11.

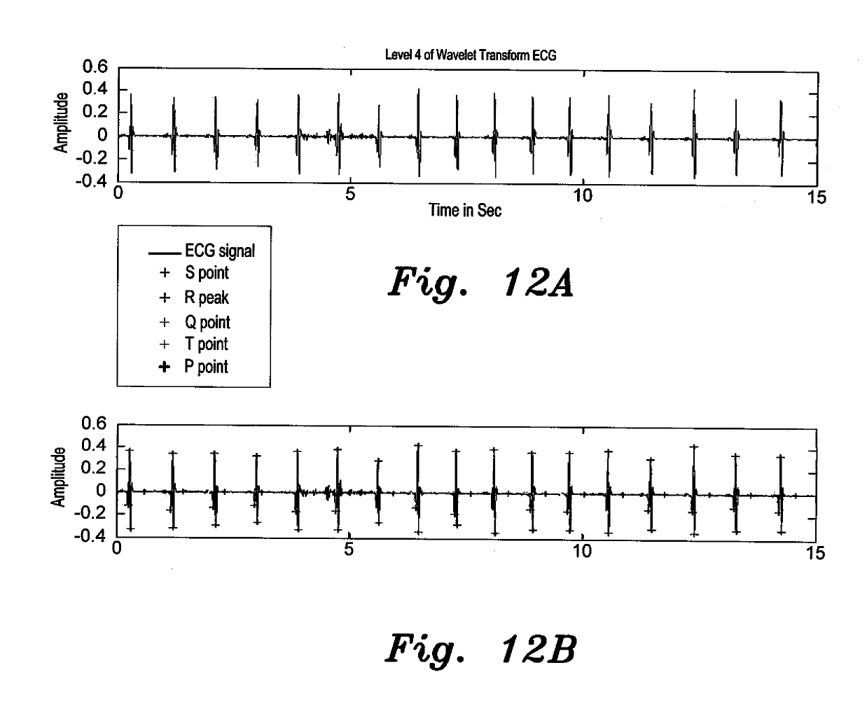

After producing the filtered and baseline wander corrected electrocardiogram signal, the continuous wavelet transformation is performed on the signal. The detection of the R peak is based on the threshold level to calculate the maximum amplitude in the ECG waveform. The R peak detection is performed in the time scale domain at scale 2⁴, shown in FIG. 12A. This same scale level is used to detect other key points in the ECG waveform.

The P, Q, S and T waves are then detected using the Tompkins method. After detecting the R peak, the first inflection points to the left and right are estimated as the Q and S peaks, respectively. After estimating the S-point, the J-point was estimated to be the first inflection point after the S-point to the right of the R peak. The T peak was estimated to be between the R peak+400 ms to the J-point+80 ms. Similarly, the K-point was estimated to be the first inflection point after the Q peak on the left side of the R peak, and the P-point was estimated to be the first inflection point after the K-point on the P peak side. The detected P, Q, R, S and T peaks are shown in FIG. 12B.

Once the P, Q, R, S and T peaks are determined, the R-R interval mean (where the R-R interval is the interval between one R wave and the next R wave); the R-R interval variance; the P height mean; the P-R duration; and the Q-T duration are calculated. This five-feature set is used for classification of the given ECG signal in seizure or non-seizure groups by the classifier, which will be described in detail below.

After the features of interest are determined, the EEG signals are classified into seizure and non-seizure traces. For this purpose, two different classifier techniques are used. The first technique is linear discriminant analysis (LDA) and the second technique is the Naïve Bayesian Classifier (NBC), which is a simple Bayesian classifier based on Bayes theorem, which considers all events to be conditionally independent of one another. Linear discriminant analysis is one of the most commonly used dimension reduction techniques, which was originally used for dimensionality reduction by projecting high-dimensional data onto a low-dimensional space, where the data achieves maximum class separability. The resulting features in LDA are linear combinations of the original features, where the coefficients are obtained using a projection matrix W. The optimal projection or transformation is obtained by minimizing within-class-distance (i.e., between the signals of the same group) and maximizing between-class-distance (i.e., between the signals belonging to different groups) simultaneously, thus achieving maximum class discrimination. The optimal transformation is readily computed by solving a generalized eigenvalue problem.

The initial LDA formulation, known as Fisher Linear Discriminant Analysis (FLDA), was originally developed for binary classifications. The focus in FLDA is to look for a direction that separates the class means well (when projected onto that direction) while achieving a small variance around the means. Discriminant analysis is generally used to find a subspace with M−1 dimensions for multi-class problems, where M is the number of classes in the training dataset.

More formally, for the available samples from the database, two measures are defined: the within-class scatter matrix and the between-class scatter matrix. The within-class scatter matrix is given by:

$${S}_{W} = {\sum }_{j = 1}^{M} {\sum }_{i = 1}^{{N}_{i}} ( {x}_{i}^{j} - {μ}_{j} ) {( {x}_{i}^{j} - {μ}_{j} )}^{T} , \qquad ( 3 )$$

where xᵢ^{j} is the i-th sample vector of class j (having a dimension of n×1), μⱼ is the mean of class j, M is the number of classes, and Nᵢ is the number of samples in class j. The between-class scatter matrix is defined as:

$${S}_{b} = {\sum }_{j = 1}^{M} ( {μ}_{j} - μ ) {( {μ}_{j} - μ )}^{T} , \qquad ( 4 )$$

where μ is the mean vector of all classes.

The goal in LDA is to find a transformation W that maximizes the between-class measure, while minimizing the within-class measure. One way to do this is to maximize the ratio det(S_{b})/det(S_{w}). The advantage of using this ratio is that if S_{w} is a non-singular matrix, then this ratio is maximized when the column vectors of the projection matrix W are the eigenvectors of S_{w}⁻¹·S_{b}. It should be noted that there are, at most, M−1 nonzero generalized eigenvectors. Thus, there is an upper bound of reduced dimension, namely M−1. Further, at least n (the size of the original feature vectors)+M samples are required to guarantee that S_{w} does not become singular.

LDA is used here to classify the features obtained from the above method in two different groups, namely “seizure” and “non-seizure”. The LDA algorithm initially assigns a group to a set of features belonging to the same class, and when the algorithm is trained with the set of features available for training, it classifies the test vector features to one of the groups using Euclidean distance as a measure to know which group the given signal belongs to. In the present method, LDA is used to perform classification of the features obtained for both EEG and ECG signals. The LDA is applied individually to both the EEG and ECG seizure detection techniques, and the results of the individual classifiers are discussed below.

The naïve Bayesian classifier is a simple form of the Bayesian classifier that is used to reduce the computational complexities that arise in the application of Bayesian classifiers applied to large feature sets. A Bayesian classifier is a statistical classifier that predicts the probability of the association of a feature to one of the classes assigned in the training feature set. The naïve Bayesian classifier is a special case of a simple Bayesian classifier that assumes that the effect of individual feature sets on the output class is independent of one another. This assumption is called “class conditional independence” and simplifies the original Bayesian classifier, hence the name “naïve” Bayesian classifier.

A simple Bayesian classifier uses the Bayes theorem, which is generally stated as follows: Let X be a feature set of X=[x₁, x₂, . . . xₙ] and let K be a hypothesis of X belonging to class Cᵢ, which is the classification goal, given by P(K=Cᵢ/X), then finding the probability of a particular feature belonging to class Cᵢ given the feature set X is given by:

$$P ( K = {C}_{i} \text{/} X ) = \frac{P ( X \text{/} {C}_{i} ) \cdot P ( C )}{P ( X )} , \qquad ( 5 )$$

where P(C) is the probability of the number of classes assigned in the feature set, which is a priori probability, P(X) is the probability of occurrence of the feature and is the same for all classes, and P(X/Cᵢ) is the probability of feature set X, given the class of the feature Cᵢ, which is a posteriori probability.

These probabilities can be easily estimated from the given data. The sample feature vector X=[x₁, x₂, . . . xₙ] is grouped and assigned to respective classes C, depending on the requirements, and are denoted by C=[C₁, C₂, . . . Cᵢ]. The classifier now assigns the vector X to a particular class Cᵢ that has the highest posterior probability given the input X, i.e., the feature vector X is assigned to a particular class Cᵢ based on the following criteria:

P(Cᵢ/X)>P(Cₖ/X),where i≠k. (6)

Thus, the class for which P(Cᵢ/X) is maximum must now be found. Since it is now known that the P(Cᵢ) and P(X) are prior probabilities, and are also fixed and remain the same, the only thing that must be maximized is P(X/Cᵢ). In the naïve Bayesian classifier, the conditional probabilities class dependence is assumed to be independent of one another, which means that P(X/Cᵢ)·P(Cᵢ)≈Πₙ^{j=1}P(xⱼ/Cᵢ). With this assumption of independence in class conditional probabilities, the individual probabilities can be easily estimated from the data set by assuming the features to be continuously valued. Thus, a Gaussian distribution with a mean and distribution may be used:

$$g ( x , μ , σ ) = \frac{1}{2 πσ} exp ( - \frac{{( x - μ )}^{2}}{2 {σ}^{2}} ) . \qquad ( 7 )$$

From equation (7), the P(xⱼ/Cᵢ) can be computed as P(xⱼ/Cᵢ)=g(xⱼ,μ_{Ci},σ_{Ci}), where μ_{Ci} and σ_{Ci} are the mean and standard deviation for a particular class, respectively. This must be computed for all of the classes. The classifier assigns the test feature vector X to a particular class Cᵢ for which the P(xⱼ/Cᵢ) is maximum. The naïve Bayesian classifier is applied to both the ECG and the EEG datasets, and the results of the trained classifier are used separately for each classifier, as discussed in detail below.

From 200 sample traces, 45 sample traces from healthy individuals and 45 sample traces from subjects with seizures were used to train the LDA classifier. After estimating the LDA transformation matrix, the testing stage was initiated by projecting the test data over the LDA matrix, then using the Euclidian distances to classify a given test pattern as either a seizure or a non-seizure trace. Similarly, the traces were then used for training the naïve Bayesian classifier, and the Gaussian mean and standard deviation needed for the conditional probabilities were calculated and were tested against the training set. Accuracy was evaluated as the number of correct detections divided by the total number of traces of healthy and seizure events; the specificity was evaluated as the number of true negatives detected divided by the number of true negatives and the number of false positives; and the sensitivity was evaluated as the number of true positives detected divided by the number of true positives and the number of false negatives.

The specificity of a classifier of 100% means that the classifier identifies all healthy people as healthy, whereas a sensitivity of 100% means that the classifier identifies all sick people as sick. The detection accuracy may also be specified in terms of good detection rate (GDR) and false detection rate (FDR). The GDR is given by GDR=100×GD/R, and the FDR is given by FDR=100×FD/(GD+FD), where GD and FD are the total number of good detections and false detections, respectively, and R is the total number of seizures correctly recognized by a neurologist. It can be seen that the detection accuracy is dependent on the accuracy of the neurologist in predicting a seizure from the raw EEG data. It has been found that the expert neurologist reports in the past were 94% accurate.

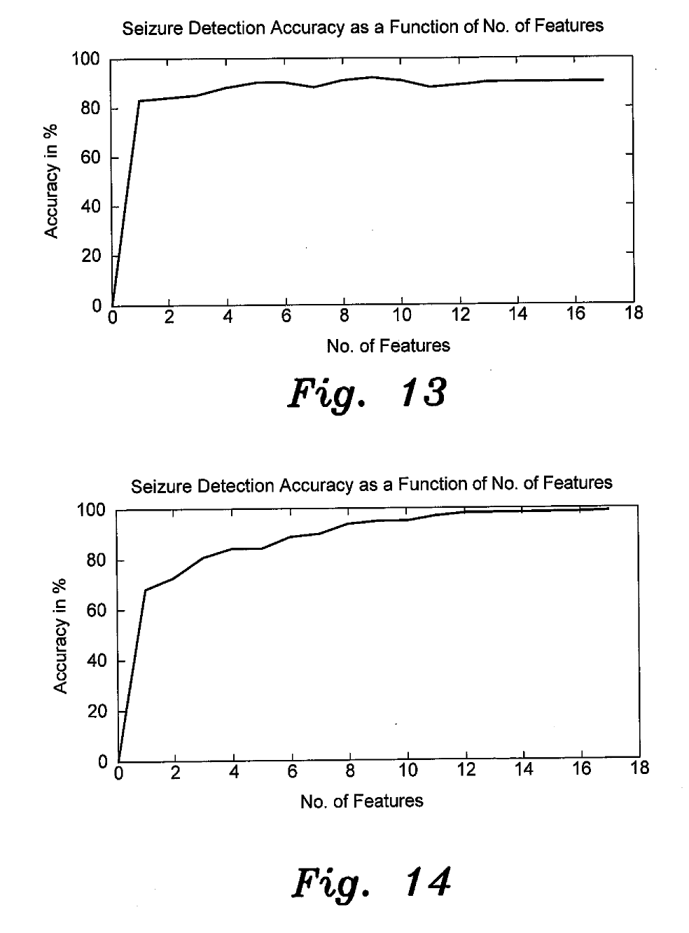

Out of the 110 EEG samples tested, an average accuracy of correct classification of 90% was achieved with LDA, and an average accuracy of 97.81% was achieved using the naïve Bayesian classifier. The experiment was carried out by randomly selecting different sets for testing and training. The recognition rates obtained for ten trials were all very close to 90% (between 87% and 95%) using LDA, and 97.81% (between 96% and 99%) with the naïve Bayesian classifier. For a given dataset, FIGS. 13 and 14 show the changes in seizure detection accuracy as the number of features used in the LDA and naïve Bayesian classifier are varied, respectively. It should be noted that around ten features are largely sufficient to represent the variations in the data for both of the classifiers.

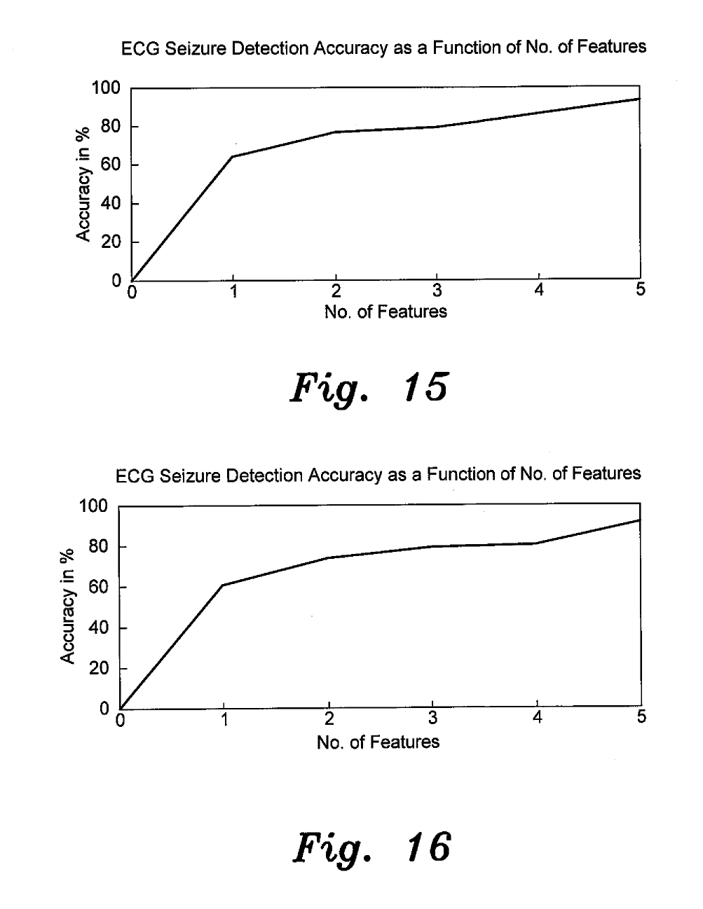

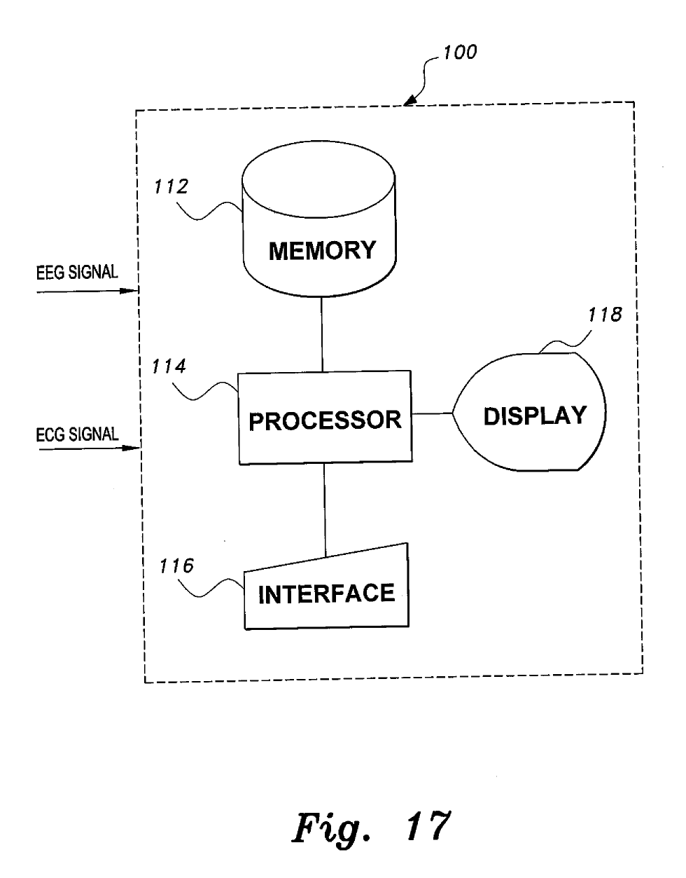

For ECG data, 55 observations of seizures and 55 observations of non-seizure intervals were used. As with the EEG data, the ECG data was tested using both LDA and the naïve Bayesian classifier. Accuracy was found to be about 93.23% and 94.81%, respectively. The variation of accuracy of the classifier with respect to the features is shown in FIGS. 15 and 16 for LDA and the naïve Bayesian classifier, respectively.

The present method uses Dempster-Shafer Theory (DST), a well-known theory of evidence, for the combination of individual LDA or naïve Bayesian classifiers. DST is used because of its ability to model the uncertainty present in the classifiers. The two types of uncertainty generally associated with any system are aleatory uncertainty (the uncertainty which results from the fact that the system can behave in random ways, such as noise) and epistemic uncertainty (the uncertainty resulting from a lack of knowledge about a system; i.e., a type of subjective uncertainty).

Aleatory uncertainty is generally overcome by using the frequentist approach associated with traditional probability. Thus, the major problem lies with epistemic uncertainty, which represents a lack of knowledge related to some event. In probability theory, it is necessary to have knowledge of all types of events. When this is not available, a uniform distribution function is often used, i.e., it is assumed that all simple events for which a probability distribution is not known in a given sample space are equally likely. An additional axiom of the Bayesian theory is that the sum of the belief and disbelief in an event should add to 1; i.e., P(x)+P( x̅)=1. The Dempster-Shafer theory of evidence rejects this axiom outwardly and introduces the concept of “beliefs”, allowing for the combination of evidence obtained from multiple sources and modelling of conflicts between them.

As an example, let Φ represent an exemplary statement, “the place is beautiful.” Then, according to the Bayesian theorem, P(Φ)+P( Φ̅)=1 where Φ̅ represents negation of the proposed statement. Considering a person X, who has never visited the place at all, and thus has no idea about what the place looks like, person X cannot say that he has belief in the above statement. Obviously, this represents not only an uncertainty in the situation, but also a limitation in Bayesian theory. Dempster-Shafer theory, on the other hand, notes the belief of the person X in the given statement, m(Φ)=0, and his disbelief, m( Φ̅)=0, indicating that the person X is uncertain of the event.

Thus, the major difference between the Bayesian formulation and Dempster-Shafer theory, when it comes to actual solutions, is conceptual. The statistical model assumes that there exist Boolean phenomena, whereas DST deals with a “belief” in that particular event. The result of the Bayesian formulation leads to the assumption that commitment in belief of a certain hypothesis leads to the commitment of the remaining belief to its negation. Thus, if one believes in the existence of a certain hypothesis, this would imply, under the Bayesian formulation, a large belief in its non-existence, which is referred to as “over-commitment”. In DST, one considers the evidence in favor of hypothesis. There is no causal relationship between a hypothesis and its negation, rather a lack of belief in any particular hypothesis implies belief in the set of all hypotheses, which is referred to as the “state of uncertainty”. If the uncertainty is denoted by θ, then, for the above example, m(θ)=1, which is calculated as: m(Φ)+m( Φ̅)+m(θ)=1.

In DST, a “basic belief assignment” (BBA) is the basis of evidence theory. It assigns a value between 0 and 1 to all of the variables in a subset A, where the BBA of the null set is 0 and the summation of BBAs of all subsets should be equal to 1. The BBA is represented by the operator b. Thus, the above may be stated as:

b(φ)=0; and Σ_{A⊂θ}b(A)=1, (8)

where φ represents the null set. The BBA b(.) for a given set U represents the amount of belief that a particular element of X (a universal set) belongs to the set U (represented by m(A)) but to no particular subset of A. The value of b(A) pertains only to set U and makes no additional claims about any subsets of A. Any further evidence on the subsets of A would be represented by another BBA b(B), where B is a subset of A.

The “belief function” in DST is used to assign a value [0, 1] to every nonempty subset B. For every probability assignment, two bounds of intervals can be defined. The lower bound in DST is represented by the belief function. This is defined as the sum of all of the basic belief assignments (BBAs) of the proper subsets of B of the set of interest A (B⊂A). This is called the “degree of belief” (represented by the “Bel” operator) in B and is defined by:

Bel(A)=Σ_{B⊂A}b(B), (9)

where B is a subset of A. The belief function can be considered as a generalization of the probability distribution function, whereas the basic belief assignment can be considered as a generalization of the probability density function.

In DST, the upper limit of the probability assignment is called the “plausibility”. The plausibility (represented by the operator “Pl”) is the sum of all of the probability assignments of the sets B that intersect the set of interest A (B∩A≠Φ):

$$Pl ( A ) = {\sum }_{B / B ⋂ A \neq ϕ} b ( B ) . \qquad ( 10 )$$

The belief and plausibility measures represent the lower and upper bound of probability for a given hypothesis, respectively. These two measures are non-additive, since the sum of all belief functions or the sum of all plausibility functions is not necessarily equal to 1.

The “combination rule” in DST theory depends on the basic belief assignments b(.). Letting b₁(.) and b₂(.) be two basic belief assignments for the belief function Bel₁(.) and Bel₂(.), respectively, and letting these two belief functions be the focal elements of the sets Bⱼ and Cₖ, respectively, then the combined belief committed to A⊂θ is given by:

$${b}_{12} ( A ) = \frac{{Σ}_{B ⋂ C = A} {b}_{1} ( B ) {b}_{2} ( C )}{1 - K} , \qquad ( 11 )$$

when A≠φ, and where K=1−Σ_{B∩C=Ø}b₁(B)b₂(C). The variable K represents the basic probability mass and is associated with conflict. The entire term 1−K represents the normalizing factor, which has the effect of completely ignoring the effect of conflict and attributing any probability mass associated with conflict to the null set.

The combination of results from both classifiers is performed using the Dempster-Shafer rule. For this, the information available from the ECG and EEG datasets is in the form of probability information, as described above. In order to combine the classifier information for both ECG and EEG, the first step is calculating the normalized distance. Before the beliefs can be extracted, the probability information is extracted from the ECG and EEG signals. This is performed by finding the Euclidean distance between the feature vector under test and the mean of the seizure class feature vectors and the non-seizure class vectors as ν=(x−μ)/σ, where x is the test feature vector, μ is the mean of the class feature vectors, and σ is the variance of the class feature vectors. The Euclidean distance v is substituted into the normal distribution to get the probability value for seizure and the probability value of non-seizure of an event.

From the probability information, the basic belief is calculated. The probability of a seizure event is assumed to be the belief in a seizure event, and the probability of a normal case is considered to be the belief in non-seizure. The conflict between the two probability values is considered as the uncertainty of information. From this basic belief, the belief and plausibility of the event is calculated. This is calculated as Bel(p)=1−Pl( p̅), where the belief represents the minimum probability of the happening of an event, and the plausibility represents the maximum amount of probability of the happening of the event.

The resulting belief functions are then combined using DST as:

$${b}_{12} ( A ) = \frac{{Σ}_{B ⋂ C = A} {b}_{1} ( B ) {b}_{2} ( C )}{1 - K} , \qquad ( 12 )$$

when A≠φ and where K=1−Σ_{B∩c=Ø}b₁(B)b₂(C), and 1−k represents the normalizing factor. The resultant belief is then compared against a threshold value of ½. When the belief probability is above ½, it is determined that a seizure event is occurring, and when the belief probability is below ½, it is determined that the event is a non-seizure event.

To test the above method, 90 sample EEG traces and 110 ECG traces were used for training (case 1). The results are shown below in Table 3.

TABLE 3
Combination of EEG and ECG Signals using DST (Case 1)
D-S Theory with LDA Classifiers

| | | | Combination |
|---|---|---|---|
| | | | using D-S Theory |
| Measure | ECG Using LDA | EEG Using LDA | of Evidence |
| Accuracy | 93.23% | 90.00% | 96.90% |
| Sensitivity | 96.49% | 92.50% | 95.87% |
| Specificity | 89.97% | 87.50% | 97.93% |

D-S Theory with Naïve Bayesian Classifier

| | | | Combination |
|---|---|---|---|
| | ECG using Naïve | EEG using Naïve | using D-S Theory |
| Measure | Bayesian | Bayesian | of Evidence |
| Accuracy | 94.81% | 97.81% | 99.00% |
| Sensitivity | 96.72% | 98.00% | 99.09% |
| Specificity | 92.90% | 97.63% | 98.90% |

As shown in Table 3, classification using the naïve Bayesian classifier provides a higher degree of accuracy (close to 100%) than use of the LDA classifier. Table 4 shows the results for a case in which five non-seizure traces and five seizure traces were added (case 2). For individual detection from either ECG or EEG classifiers, this results in a decrease of accuracy. However, as shown below, using the DST for the combination of classifiers gives an accuracy of 90.74% for LDA classifiers and 93.18% for naïve Bayesian classifiers.

TABLE 4
Combination of EEG and ECG Signals using DST (Case 2)
D-S Theory with LDA Classifiers

| | | | Combination |
|---|---|---|---|
| | | | using D-S Theory |
| Measure | ECG Using LDA | EEG Using LDA | of Evidence |
| Accuracy | 75.83% | 84.16% | 90.74% |
| Sensitivity | 78.94% | 86.50% | 93.64% |
| Specificity | 72.72% | 81.82% | 88.02% |

D-S Theory with Naïve Bayesian Classifier

| | | | Combination |
|---|---|---|---|
| | ECG using Naïve | EEG using Naïve | using D-S Theory |
| Measure | Bayesian | Bayesian | of Evidence |
| Accuracy | 81.18% | 88.27% | 93.18% |
| Sensitivity | 82.00% | 89.45% | 93.63% |
| Specificity | 80.36% | 87.09% | 93.72% |

The data used for EEG and ECG each belong to different databases, thus, in order to show the degree of association between the two different databases, a test was performed. A database of 90 ECG/EEG traces was used for testing, and 120 ECG/EEG traces were used for training. It is assumed that person X's ECG corresponds to person Y's EEG. To show the degree of association, 10 samples of the EEG database were shifted each time and associated with the ECG database. At each shift, the detection accuracy of the algorithm was measured. The effect of this shift on the combination accuracy for cases 1 and 2 are shown in Tables 5 and 6 below.

TABLE 5
Degree of Association for Case 1

| | D-S Theory | | | D-S Theory of Evidence with | | |
|---|---|---|---|---|---|---|
| | of Evidence with LDA | | | Naïve Bayesian | | |
| | | Sensi- | | | Sensi- | |
| Shift | Accuracy | tivity | Specificity | Accuracy | tivity | Specificity |
| 1^{st} | 96.70% | 96.25% | 97.15% | 100.00% | 100% | 100% |
| 2^{nd} | 95.20% | 94.62% | 95.78% | 99.09% | 98.18% | 100% |
| 3^{rd} | 98.30% | 96.68% | 99.92% | 99.09% | 98.18% | 100% |
| 4^{th} | 96.70% | 95.83% | 97.57% | 99.09% | 100% | 98.18% |
| 5^{th} | 98.36% | 97.24% | 99.48% | 100% | 100% | 100% |
| 6^{th} | 96.70% | 95.00% | 98.40% | 96.36% | 98.18% | 94.54% |
| 7^{th} | 95.20% | 94.16% | 96.24% | 99.09% | 98.18% | 100% |
| 8^{th} | 98.30% | 97.45% | 99.15% | 99.09% | 100% | 98.18% |
| 9^{th} | 95.23% | 94.28% | 96.18% | 100% | 100% | 100% |
| 10^{th} | 98.36% | 97.24% | 99.48% | 98.00% | 98.18% | 98.18% |

TABLE 6
Degree of Association for Case 2

| | D-S Theory | | | D-S Theory of Evidence with | | |
|---|---|---|---|---|---|---|
| | of Evidence with LDA | | | Naïve Bayesian | | |
| | | Sensi- | | | Sensi- | |
| Shift | Accuracy | tivity | Specificity | Accuracy | tivity | Specificity |
| 1^{st} | 92.34% | 95.23% | 89.45% | 94.54% | 96.36% | 92.72% |
| 2^{nd} | 90.83% | 90.90% | 90.76% | 89.09% | 94.54% | 83.63% |
| 3^{rd} | 86.56% | 93.70% | 79.42% | 90.90% | 90.90% | 90.90% |
| 4^{th} | 91.66% | 92.30% | 91.02% | 96.36% | 92.72% | 100% |
| 5^{th} | 89.25% | 93.70% | 84.80% | 93.63% | 96.36% | 90.90% |
| 6^{th} | 93.84% | 95.23% | 92.45% | 93.63% | 96.36% | 90.90% |
| 7^{th} | 92.50% | 95.23% | 89.77% | 94.54% | 90.90% | 98.18% |
| 8^{th} | 90.83% | 90.90% | 90.76% | 92.72% | 90.90% | 94.54% |
| 9^{th} | 90.75% | 93.75% | 87.75% | 90.90% | 92.72% | 89.09% |
| 10^{th} | 88.89% | 93.70% | 84.08% | 95.45% | 94.54% | 96.36% |

It should be understood that the calculations may be performed by any suitable computer system, such as that diagrammatically shown in FIG. 17. Data is entered into controller 100 via any suitable type of user interface 116, and may be stored in memory 112, which may be any suitable type of computer readable and programmable memory and is preferably a non-transitory, computer readable storage medium. Calculations are performed by processor 114, which may be any suitable type of computer processor and may be displayed to the user on display 118, which may be any suitable type of computer display.

Processor 114 may be associated with, or incorporated into, any suitable type of computing device, for example, a personal computer or a programmable logic controller. The display 118, the processor 114, the memory 112 and any associated computer readable recording media are in communication with one another by any suitable type of data bus, as is well known in the art.

Examples of computer-readable recording media include non-transitory storage media, a magnetic recording apparatus, an optical disk, a magneto-optical disk, and/or a semiconductor memory (for example, RAM, ROM, etc.). Examples of magnetic recording apparatus that may be used in addition to memory 112, or in place of memory 112, include a hard disk device (HDD), a flexible disk (FD), and a magnetic tape (MT). Examples of the optical disk include a DVD (Digital Versatile Disc), a DVD-RAM, a CD-ROM (Compact Disc-Read Only Memory), and a CD-R (Recordable)/RW. It should be understood that non-transitory computer-readable storage media include all computer-readable media, with the sole exception being a transitory, propagating signal.

It is to be understood that the present invention is not limited to the embodiments described above, but encompasses any and all embodiments within the scope of the following claims.

We claim:

1. A method for detecting seizure activity, comprising the steps of:
receiving an electroencephalogram signal taken from a patient;
representing the electroencephalogram signal in a time-frequency domain;
generating a time-frequency representation matrix of the EEG signal;
applying singular value decomposition to the time-frequency representation matrix to compute left and right singular vectors and a singular value matrix;
extracting a set of probability mass functions from the singular value matrix;
generating a histogram having 17 bins for the left singular vector for the first singular value;
receiving an electrocardiogram signal taken from the patient;
filtering and correcting the electrocardiogram signal for baseline wander to produce a filtered and baseline wander-corrected electrocardiogram signal;
determining an R wave peak in the filtered and baseline wander-corrected electrocardiogram signal;
determining P, Q, S and T wave peaks in the filtered and baseline wander-corrected electrocardiogram signal;
calculating an R-R interval mean as a mean value between consecutive R wave peaks in the filtered and baseline wander-corrected electrocardiogram signal;
calculating an R-R interval variance as a variance between consecutive R wave intervals in the filtered and baseline wander-corrected electrocardiogram signal;
calculating a P height mean as a mean value of P wave peaks in the filtered and baseline wander-corrected electrocardiogram signal;
calculating a P-R duration as a duration between consecutive P and R wave peaks in the filtered and baseline wander-corrected electrocardiogram signal;
calculating a Q-T duration as a duration between consecutive Q and T wave peaks in the filtered and baseline wander-corrected electrocardiogram signal;
applying an electroencephalogram classifier to the histogram to calculate an electroencephalogram probability of a seizure classification;
applying an electrocardiogram classifier to a feature dataset including the R wave peak, the P, Q, S and T wave peaks, the R-R interval mean, the R-R interval variance, the P height mean, the P-R duration and the Q-T duration to calculate an electrocardiogram probability of a seizure classification;
combining the electroencephalogram probability of a seizure classification and the electrocardiogram probability of a seizure classification to determine a Dempster-Shafer belief; and
determining if the Dempster-Shafer belief has a probability value above a threshold value; and
indicating presence of a seizure event when the Dempster-Shafer belief has a probability value above the threshold value.

2. The method for detecting seizure activity as recited in claim 1, further comprising the step of filtering the electroencephalogram signal prior to representing the electroencephalogram signal in the time-frequency domain.

3. The method for detecting seizure activity as recited in claim 1, wherein the step of filtering the electrocardiogram signal comprises:
passing the electrocardiogram signal through a finite impulse response filter to generate a first filtered electrocardiogram signal;
passing the first filtered electrocardiogram signal through a median filter having a 200 ms duration to remove QRS complexes therefrom to generate a second filtered electrocardiogram signal;
passing the second filtered electrocardiogram signal through a median filter having a 600 ms duration to remove a T wave therefrom to generate a third filtered electrocardiogram signal; and
subtracting the third filtered electrocardiogram signal from the first filtered electrocardiogram signal to produce the filtered and baseline wander-corrected electrocardiogram signal.

4. The method for detecting seizure activity as recited in claim 1, wherein the step of applying the electroencephalogram classifier to the histogram comprises applying a linear discriminant analysis classifier to the histogram.

5. The method for detecting seizure activity as recited in claim 1, wherein the step of applying the electroencephalogram classifier to the histogram comprises applying a naïve Bayesian classifier to the histogram.

6. The method for detecting seizure activity as recited in claim 1, wherein the step of applying the electrocardiogram classifier to the feature dataset comprises applying a linear discriminant analysis classifier to the feature dataset.

7. The method for detecting seizure activity as recited in claim 1, wherein the step of applying the electrocardiogram classifier to the feature dataset comprises applying a naïve Bayesian classifier to the feature dataset.

8. The method for detecting seizure activity as recited in claim 1, wherein the step of combining the electroencephalogram probability of a seizure classification and the electrocardiogram probability of a seizure classification to determine the Dempster-Shafer belief is performed using the Dempster-Shafer rule.

9. The method for detecting seizure activity as recited in claim 8, wherein the step of combining the electroencephalogram probability of a seizure classification and the electrocardiogram probability of a seizure classification to determine the Dempster-Shafer belief comprises:
establishing a feature vector from the electroencephalogram probability of a seizure classification and the electrocardiogram probability of a seizure classification; and
calculating a Euclidean distance between the feature vector and a mean of a set of trained seizure class feature vectors and a set of trained non-seizure class feature vectors.

10. The method for detecting seizure activity as recited in claim 9, wherein the step of determining if the Dempster-Shafer belief has a probability value above the threshold value comprises determining if the Dempster-Shafer belief has a probability value above ½.

11. A system for detecting seizure activity, comprising:
an electroencephalogram for receiving an electroencephalogram signal taken from a patient;
an electrocardiogram for receiving an electrocardiogram signal taken from the patient;
means for representing the electroencephalogram signal in a time-frequency domain;
means for generating a time-frequency representation matrix of the electroencephalogram signal;
means for applying singular value decomposition to the time-frequency representation matrix to compute left and right singular vectors and a singular value matrix;
means for extracting a set of probability mass functions from the singular value matrix;
means for generating a histogram having 17 bins for the left singular vector for a first singular value;
means for filtering and correcting the electrocardiogram signal for baseline wander to produce a filtered and baseline wander-corrected electrocardiogram signal;
means for determining an R wave peak in the filtered and baseline wander-corrected electrocardiogram signal;
means for determining P, Q, S and T wave peaks in the filtered and baseline wander-corrected electrocardiogram signal;
means for calculating an R-R interval mean as a mean value between consecutive R wave peaks in the filtered and baseline wander-corrected electrocardiogram signal;
means for calculating an R-R interval variance as a variance between consecutive R wave intervals in the filtered and baseline wander-corrected electrocardiogram signal;
means for calculating a P height mean as a mean value of P wave peaks in the filtered and baseline wander-corrected electrocardiogram signal;
means for calculating a P-R duration as a duration between consecutive P and R wave peaks in the filtered and baseline wander-corrected electrocardiogram signal;
means for calculating a Q-T duration as a duration between consecutive Q and T wave peaks in the filtered and baseline wander-corrected electrocardiogram signal;
means for applying an electroencephalogram classifier to the histogram to calculate an electroencephalogram probability of a seizure classification;
means for applying an electrocardiogram classifier to a feature dataset including the R wave peak, the P, Q, S and T wave peaks, the R-R interval mean, the R-R interval variance, the P height mean, the P-R duration and the Q-T duration to calculate an electrocardiogram probability of a seizure classification;
means for combining the electroencephalogram probability of a seizure classification and the electrocardiogram probability of a seizure classification to determine a Dempster-Shafer belief; and
means for determining if the Dempster-Shafer belief has a probability value above a threshold value; and
means for indicating presence of a seizure event when the Dempster-Shafer belief has a probability value above the threshold value.

12. The system for detecting seizure activity as recited in claim 11, further comprising means for filtering the electroencephalogram signal.

13. The system for detecting seizure activity as recited in claim 11, wherein the means for filtering the electrocardiogram signal comprises:
a finite impulse response filter to generate a first filtered electrocardiogram signal;
a first median filter having a 200 ms duration to remove QRS complexes from the first filtered electrocardiogram signal to generate a second filtered electrocardiogram signal;
a second median filter having a 600 ms duration to remove a T wave from the second filtered electrocardiogram signal to generate a third filtered electrocardiogram signal; and
means for subtracting the third filtered electrocardiogram signal from the first filtered electrocardiogram signal to produce the filtered and baseline wander corrected electrocardiogram signal.

14. The system for detecting seizure activity as recited in claim 11, wherein the means for applying the electroencephalogram classifier to the histogram includes a linear discriminant analysis classifier.

15. The system for detecting seizure activity as recited in claim 11, wherein the means for applying the electroencephalogram classifier to the histogram includes a naïve Bayesian classifier.

16. The system for detecting seizure activity as recited in claim 11, wherein the means for applying the electrocardiogram classifier to the feature dataset applies a linear discriminant analysis classifier to the feature dataset.

17. The system for detecting seizure activity as recited in claim 11, wherein the means for applying the electrocardiogram classifier to the feature dataset applies a naïve Bayesian classifier to the feature dataset.

18. The system for detecting seizure activity as recited in claim 11, wherein the means for combining the electroencephalogram probability of a seizure classification and the electrocardiogram probability of a seizure classification to determine the Dempster-Shafer belief applies the Dempster-Shafer rule.

19. The system for detecting seizure activity as recited in claim 18, wherein the means for combining the electroencephalogram probability of a seizure classification and the electrocardiogram probability of a seizure classification to determine the Dempster-Shafer belief comprise:
means for establishing a feature vector from the electroencephalogram probability of a seizure classification and the electrocardiogram probability of a seizure classification; and
means for calculating a Euclidean distance between the feature vector and a mean of a set of trained seizure class feature vectors and a set of trained non-seizure class feature vectors.

20. The system for detecting seizure activity as recited in claim 19, wherein the threshold value is equal to ½.
